(19) United States
(12) Patent Application Publication
(10) Pub. No.: US 2021/0407637 A1
(43) Pub. Date: Dec. 30, 2021

(54) METHOD TO DISPLAY LESION READINGS RESULT

(71) Applicant: VUNO Inc., Seoul (KR)

(72) Inventors: Beomhee PARK, Seoul (KR); Woong BAE, Seoul (KR); Minki CHUNG, Seoul (KR)

(21) Appl. No.: 17/356,111

(22) Filed: Jun. 23, 2021

(30) Foreign Application Priority Data
Jun. 24, 2020 (KR) 10-2020-0077352

(51) Int. Cl. G16H 15/00; G06T 7/00; G16H 50/20; G16H 70/60; G16H 30/40; G16H 50/70; G16H 10/60

(52) U.S. Cl. CPC G16H 15/00; G06T 7/0012; G16H 50/20; G06F 3/04842; G16H 30/40; G16H 50/70; G16H 10/60; G16H 70/60

(57) ABSTRACT

Disclosed is a computer program stored in a computer readable storage medium. When the computer program is executed by one or more processors, the computer program provides a user interface for displaying a lesion readings result, and the user interface may include: lesion information comprised in medical data; and one or more findings related to the lesion information and displayed in response to a user interaction with the lesion information.

### CROSS-REFERENCE TO RELATED APPLICATIONS

This application claims priority to and the benefit of Korean Patent Application No. 10-2020-0077352 filed in the Korean Intellectual Property Office on Jun. 24, 2020, the entire contents of which are incorporated herein by reference.

### BACKGROUND

### Technical Field

The present disclosure relates to a method to display a lesion readings result, and more particularly, to a method to display lesion information included in medical data.

### Description of the Related Art

Medical images make it possible to identify a patient's body inside, thereby significantly helping a patient diagnosis of physicians. For example, it is possible to check whether the heart, the lung, the bronchus, or the like is abnormal through the medical images.

However, in the case of some medical images, there is a high read difficulty, even in the case of medical teams, which are experienced for years, it is difficult to decide the heart, the lung, the bronchus, or the like is abnormal rapidly.

Korean Patent Unexamined Publication No. 2019-0105461 discloses a computer assist diagnosis system.

### BRIEF SUMMARY

The inventors of the present disclosure have identified that in the case of a lung CT image, there are many kinds of nodules, and the reading difficulty for such nodules is significant in the related art. In addition, the inventors have identified that there is a probability of ignoring very fine abnormalities included in the medical image when the abnormalities are read by a person. The inventors have recognized that there is a demand in the related art for assisting medical image reading of the physicians and the inventors have provided one or more embodiments that addresses one or more problems in the related art.

Among other technical benefits, the present disclosure has been made in an effort to provide a method to display a lesion reading result.

In an embodiment of the present disclosure, when the computer program stored in a computer-readable storage medium is executed by one or more processors, the computer program provides a user interface (UI) for displaying a lesion readings result, and the user interface may comprise: lesion information comprised in medical data; and one or more findings related to the lesion information and displayed in response to a user interaction with the lesion information.

In an alternative embodiment, the lesion information comprised in medical data may comprise: lesion information for at least one of a certain lesion in which at least some regions included in the medical data are classified as one finding, or an uncertain lesion in which at least some regions included in the medical data are not classified as one finding.

In an alternative embodiment, the certain lesion and the uncertain lesion may be distinguished and displayed.

In an alternative embodiment, the user interface may further comprise a degree of uncertainty for each of one or more findings associated with the uncertain lesion, which is a probability that the uncertain lesion can be classified as a finding associated with the uncertain lesion.

In an alternative embodiment, two or more findings of the uncertain lesion may correspond to each of the two or more classes.

In an alternative embodiment, one or more findings of the uncertain lesion may correspond to at least one of a class having a score value equal to or greater than a predetermined (or selected) second threshold value, or a class having a predetermined (or selected) higher number of score values.

In an alternative embodiment, when the at least some regions are calculated using a diagnostic model comprising one or more network functions, the uncertain lesion may comprise the at least some regions, which are not determined as one class or are determined as two or more classes, based on score values for two or more classes included in a result of the calculation.

In an alternative embodiment, when the at least some regions are calculated using a diagnostic model including one or more network functions, and based on the score values for two or more classes included in the result of the calculation, the uncertain lesion may be at least one of the following cases where there are two or more classes having a score value equal to or greater than a first threshold value, where there is no class having a score value equal to or greater than the first threshold value, where a difference between the largest score value and the other score values is less than a threshold ratio or a threshold difference value, or where a variance of score values is less than a threshold variance value.

In an alternative embodiment, when the at least some regions are calculated using a diagnostic model comprising one or more network functions, the certain lesion may comprise the at least some regions, which are determined as one class, based on the score values for two or more classes included in the result of the calculation.

In an alternative embodiment, the user interface may further comprise a degree of uncertainty for each of one or more findings associated with the uncertain lesion, which is determined according to a score value of a class corresponding to each of the one or more findings associated with the uncertain lesion.

In an alternative embodiment, the one or more findings related to the lesion information may comprise findings corresponding to a predetermined (or selected) class when a calculating result of a diagnostic model for at least some regions included in the medical data has a score equal to or greater than a third threshold value for the predetermined (or selected) class.

In an alternative embodiment, the lesion information may be displayed in different ways depending on at least one of a clinical meaning of the lesion information or a degree of uncertainty of the lesion information.

In an alternative embodiment, the lesion information may be post-processed lesion information, and the post-process method may be determined according to a user selection input or by at least one of a display of a lesion, a comparison between a lesion and a region around a lesion, or a type of findings corresponding to a lesion.

In an alternative embodiment, the one or more processors may perform an operation of generating a reading for a finding subjected to a user selection input in response to a user selection input for at least one finding related to the lesion information.

In an alternative embodiment, the user interface may not display the lesion information in response to a user deletion input for the lesion information.

In an alternative embodiment, the user interface may further comprise additional information about the lesion information, and the additional information may comprise information to aid in a judgment of a user about a lesion and at least one of patient information, history information, other medical information, or reference case information.

In an alternative embodiment, the history information regarding the medical data may comprise a comparison result of one or more lesions included in past medical data generated at a time different from the medical data and one or more lesions included in the medical data.

Another embodiment of the present disclosure provides a method for displaying a lesion readings result, which may comprise: displaying lesion information included in medical data; and displaying one or more findings related to the lesion information in response to a user interaction with the lesion information.

Still another embodiment of the present disclosure provides a server for displaying a lesion readings result, including: a processor including one or more cores; a network unit; and a memory, in which the processor may be configured to: determine to transmit a user interface (UI) to a user terminal through the network unit, and the user interface may comprise a lesion information comprised in medical data; and one or more findings related to the lesion information and displayed in response to a user interaction with the lesion information.

Yet another embodiment of the present disclosure provides a user terminal including: a processor comprising one or more cores; a memory; and an output unit providing a user interface, in which the user interface may comprise lesion information comprised in medical data; and one or more findings related to the lesion information and displayed in response to a user interaction with the lesion information.

According to an embodiment of the present disclosure, a lesion readings result can be provided.

### BRIEF DESCRIPTION OF THE SEVERAL VIEWS OF THE DRAWINGS

FIG. 1 is a block diagram of a computing device for performing an operation for displaying a lesion readings result according to an embodiment of the present disclosure.

FIG. 2 is a diagram illustrating a lesion reading result according to an embodiment of the present disclosure.

FIG. 3 is a diagram illustrating an method to read a lesion according to an embodiment of the present disclosure.

FIG. 4 is a diagram illustrating an method to read a lesion according to an embodiment of the present disclosure.

FIG. 5 is a diagram illustrating a lesion reading result according to an embodiment of the present disclosure.

FIG. 6 is a diagram illustrating a lesion readings result according to an embodiment of the present disclosure.

FIG. 7 is a diagram illustrating a lesion readings result according to an embodiment of the present disclosure.

FIGS. 8A and 8B are diagrams illustrating a lesion readings result according to an embodiment of the present disclosure.

FIG. 9 is a flowchart for showing a lesion readings result according to an embodiment of the present disclosure.

FIG. 10 is a block diagram of a computing device according to an embodiment of the present disclosure.

### DETAILED DESCRIPTION

Various embodiments will now be described with reference to the drawings. In the present specification, various descriptions are presented to provide appreciation of the present disclosure. However, it is apparent that the embodiments can be executed without the specific description.

“Component,” “module,” “system,” and the like which are terms used in the specification refer to a computer-related entity, hardware, firmware, software, and a combination of the software and the hardware, or execution of the software. For example, the component may be a processing process executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not an exclusive “or” but an inclusive “or.” That is, when not separately specified or not clear in terms of a context, a sentence “X uses A or B” is intended to mean one of the natural inclusive substitutions. That is, the sentence “X uses A or B” may be applied to any of the cases where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “unit” may include any electrical circuitry, features, components, an assembly of electronic components or the like. That is, “unit” may include any processor-based or microprocessor-based system including systems using microcontrollers, integrated circuit, chip, microchip, reduced instruction set computers (RISC), application specific integrated circuits (ASICs), field-programmable gate arrays (FPGAs), graphical processing units (GPUs), logic circuits, and any other circuit or processor capable of executing the various operations and functions described herein. The above examples are examples only, and are thus not intended to limit in any way the definition or meaning of the term “unit.”

In some embodiments, the various units described herein may be included in or otherwise implemented by processing circuitry such as a microprocessor, microcontroller, or the like.

Those skilled in the art need to additionally recognize that various illustrative logical blocks, configurations, modules, circuits, means, logic, and algorithm steps described in connection with the embodiments disclosed herein may be implemented as electronic hardware, computer software, or combinations of both sides. To clearly illustrate the interchangeability of hardware and software, various illustrative components, blocks, constituents, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented embodiments is provided so that those skilled in the art of the present disclosure use or implement the present disclosure. Various modifications to the embodiments will be apparent to those skilled in the art. Generic principles defined herein may be applied to other embodiments without departing from the scope of the present disclosure. Therefore, the present disclosure is not limited to the embodiments presented herein. The present disclosure should be analyzed within the widest range which is coherent with the principles and new features presented herein.

In an embodiment of the present disclosure, a server may include other components for performing a server environment of the server. The server may include all arbitrary types of devices. The server as a digital device may be a digital device with a calculation capability, which has a processor installed therein and a memory, such as a laptop computer, a notebook computer, a desktop computer, a web pad, or a mobile phone. The server may be a web server that processes a service. A type of server described above is just an example and the present disclosure is not limited thereto.

In the present specification, a neural network, an artificial neural network, and a network function may often be used interchangeably with each other.

The term “image” or “image data” used throughout the detailed description and claims of the present disclosure refers to multi-dimensional data constituted by discrete image elements (e.g., pixels in a 2D image), and in other words, refers to an object which may be seen with an eye (e.g., displayed on a video screen) or a digital representation of the object (such as a file corresponding to a pixel output of CT, MRI detector, etc.). For example, the “image” may be computed tomography (CT), magnetic resonance imaging (MRI), a fundus image, ultrasonic waves, a medical image of a subject collected by any other medical imaging system known in the technical field of the present disclosure. The image may not particularly be provided in a medical context, and may be provided in a non-medical context, and may be for example, a security search X-ray imaging.

Throughout the detailed description and claims of the present disclosure, a ‘Digital Imaging and Communications in Medicine (DICOM)’ standard is a term which collectively refers to several standards used for digital image representation and communication in a medical device, so that the DICOM standard is announced by the American College of Radiology (ACR) and the National Electrical Manufacturers Association (NEMA).

Throughout the detailed description and claims of the present disclosure, a Picture Archiving and Communication System (PACS)′ is a term that refers to a system for performing storing, processing, and transmitting according to the DICOM standard, and medical images such as X-ray, CT, and MM, which are acquired by using digital medical image equipment may be stored in a DICOM format and transmitted to terminals inside or outside a hospital through a network, and additionally include a reading result and a medical chart.

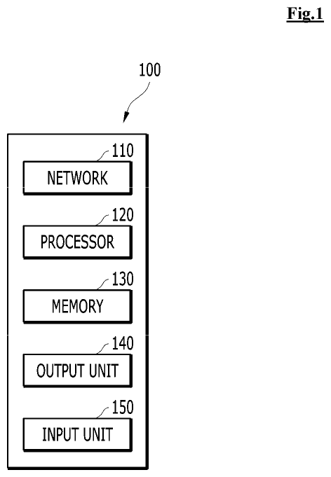

FIG. 1 is a block diagram of a computing device for performing an operation for displaying a lesion readings result according to an embodiment of the present disclosure. The lesion readings result according to an embodiment of the present disclosure may be a reading result of a lesion included in medical data.

A processor 120 may display lesion information included in the medical data.

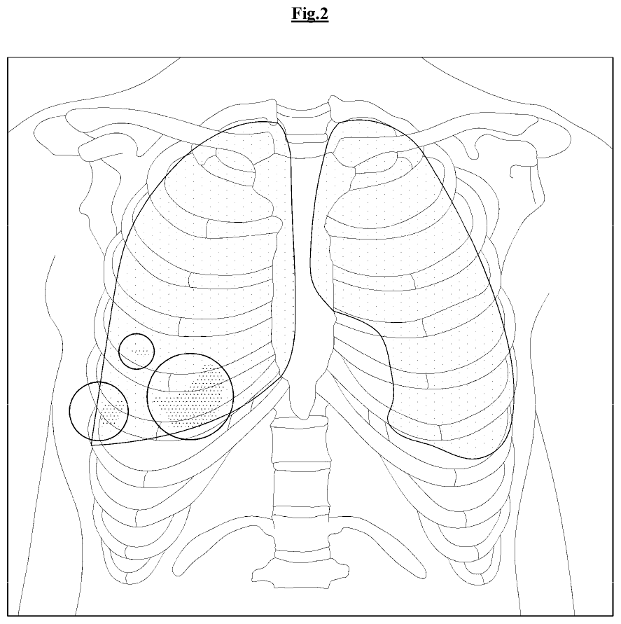

The medical data may include at least one of image data, voice data, and time-series data. That is, any type of data through which a person who works in a medical business or a device for diagnosis may determine existence of a disease in data may be included in the medical data according to the present disclosure. The image data includes all image data outputted by photographing or measuring a diseased region of a patient through inspection equipment, and converting the photographed or measured diseased region into an electrical signal. The image data may include image data constituting each frame of a moving picture in a moving picture continuously photographed according to the time from a medical image photographing device. For example, the image data includes ultrasonic inspection image data, image data by an MRI device, CT tomography image data, X-ray photographing image data, and the like. Further, when the voice data is converted into the electrical signal and output as a graph-form image or the time-series data is represented as visualized data such as a graph, etc., the corresponding image or data may be included in the image data. Referring to FIG. 2, a lung CT image illustrated in FIG. 2 may be the medical data. The above-described example of the medical data is just one example, and does not limit the present disclosure.

The lesion information may be information on a body part in which the disease appears. The lesion information may represent a position, a size, etc., of the lesion. Referring to FIG. 2, a portion indicated by a circular shape on the lung CT image may be the lesion information. The processor 120 may detect the lesion by performing a calculation by using the medical data. The processor 120 may display the lesion information on the medical data by checking the position and the size of the detected lesion. For example, for the lung CT image, the processor 120 may detect pulmonary nodule, etc., and display the lesion information in the corresponding part. A detailed description of the above-described lesion information is just an example and the present disclosure is not limited thereto.

The processor 120 may display one or more lesion information detected from the medical data on a user interface. The lesion information may be displayed in various schemes which are not limited. For example, the lesion information may be expressed as a color based heat map, a shape based heat map, etc. The color based heat map may be a method that distinguishes and displays parts having a high probability of corresponding to the lesion and parts having a low probability of corresponding to the lesion with different colors among regions of the medical data. For example, among the regions included in the medical data, a region having a high probability of belonging to the lesion may be expressed as a red color, a region having a low probability of belonging to the lesion may be expressed as a blue color, and a normal region which does not belong to the lesion may not be expressed as a color. The shape based heat map may be a method that distinguishes and displays parts having a high probability of corresponding to the lesion and parts having a low probability of corresponding to the lesion with different shapes among regions of the medical data. For example the shape based heat map may be displayed by a line, a dotted line, a double line, a dark line, a circular, a rectangle, etc. For example, among the regions included in the medical data, the region having the high probability of belonging to the lesion may be displayed by the circular, and the region having the low probability of belonging to the lesion may be displayed by the rectangle. For example, among the regions included in the medical data, the region having the high probability of belonging to the lesion may be displayed by the double line/dark line, and the region having the low probability of belonging to the lesion may be displayed by a single line/a light line. The detailed description of the display of the lesion information is just an example and the present disclosure is not limited thereto.

The processor 120 may change a lesion information display method according to a user selection input. For example, the processor 120 may display the lesion information by the color based heat map designated by a default method. In addition, when a user inputs to display the lesion information by another method, the processor 120 may display the lesion information by the corresponding method. The detailed description of the display of the lesion information is just an example and the present disclosure is not limited thereto.

The processor 120 may display the lesion information by different schemes according to findings. For example, in the lung CT image, a finding for a first region may be considered as consolidation and a finding for a second region may be determined as nodule. The processor 120 may display the lesion information for the first region by the single line and display the lesion information for the second region by the dotted line. The processor 120 may display the lesion information differently according to the finding by a predetermined (or selected) method according to the finding. Alternatively, the processor 120 may display the lesion information differently according to the finding by a method according to user setting. The detailed description of the display of the lesion information is just an example and the present disclosure is not limited thereto.

When users read a medical image having a high difficulty or read a medical image including a very small lesion, some reading results may be inaccurate. Alternatively, when medical images of unskilled users are read, some reading results may be inaccurate. According to an embodiment of the present disclosure, the medical data and the lesion information are provided to the users to assist reading of the medical data of the users. A reading result using a neural network is provided to allow the users to refer to the corresponding result.

The processor 120 may display lesion information for at least one of a certain lesion or an uncertain lesion included in the medical data. According to an embodiment of the present disclosure, the processor 120 separately displays the uncertain lesion on the user interface to assist the user to read more accurate medical data. In respect to the medical data, since there is a limit of information which modality has, it may be difficult to determine a specific lesion by one finding. In detecting the specific lesion included in the medical data, the finding may vary depending on the user who reads the corresponding medical data or the finding may vary even though the same user re-reads the corresponding medical data. For the lesion having the high uncertainty, lesion reading software distinguishes and displays the medical data by a single finding to degrade reliability for the corresponding software. Further, when the medical data is read and displayed by the single finding, an accurate reading result for the disease of the patient is not provided to cause a danger for the patient. Accordingly, when the lesion readings result is provided, a lesion having uncertainty needs to be effectively transferred to the user. According to an embodiment of the present disclosure, the lesion having the uncertainty is displayed on the user interface to decrease dangerousness of misdiagnosis and increase convenience and efficiency of reading of the medical data by the user.

Throughout the present specification, the finding may be data which becomes a basis of determination of a final disease. Various findings may be associated in generating diagnosis result information in diagnosing one disease. Further, one finding may also serve to become a basis of determination of various diseases. In general, a specific disease is diagnosed based on presence of one or more specific findings. For example, in diagnosing the disease of lung cancer, a first finding that a rale is auscultated during inhalation at portions below both lungs and a second finding that a pulmonary shade appears at a portion below the lung in thoracic CT are aggregated to make a final diagnosis. As another example, in diagnosing a disease of the glaucoma, a first finding according to a damage degree of the retinal nerve fiber layer and a second finding according to the vessel change of the optic nerve nipple periphery and the macula portion may be involved. As described above, throughout the present specification, the finding or finding information may be appreciated as an independent variable which causes the disease and diagnosis result information including the type of disease diagnosed as a result of aggregating the findings may be appreciated as a dependent variable according to the independent variable. It will be apparent to those skilled in the art that the type and finding of disease described above are just examples not to limit the present disclosure, and the embodiments of the disease and the finding need not be limited in the present disclosure for quantifying which effect the change of the independent variable exerts on the result of the dependent variable.

The certain lesion may be a lesion in which at least some regions included in the medical data are classified as one finding. The uncertain lesion may be a lesion in which at least some regions included in the medical data are not classified as one finding. That is, when the lesion included in the medical data is not classified into one finding, information on uncertainty may be provided to the user. Based on the information on the uncertainty, the user may read the medical data again, or generate (e.g., photograph the body by different schemes or re-photograph the body by the same scheme) and read the medical data again.

Hereinafter, a method for determining the certain lesion and the uncertain lesion will be described.

The processor 120 may calculate at least some regions included in the medical data by using a diagnosis model including one or more network functions. The diagnosis model may be a pre-learned neural network model. The diagnosis model may calculate the medical data as an input. The diagnosis model may output lesion information included in the input medical data and a finding therefor.

The diagnosis model may be a model that is learned by using learning data having the medical data as the input, and having lesion information and a finding included in the medical data as a label. The learning data may include medical data, and lesion information and a finding which may be detected from the medical data. For example, the learning data may include the lung CT image (e.g., medical data) as the input and a position (e.g., lesion information) which is abnormal and consolidation (e.g., finding) which is the type of abnormality included in the lung CT image as the label. The detailed description of the learning is just an example and the present disclosure is not limited thereto.

The diagnosis model may be a deep neural network. Throughout the present specification, a neural network, a network function, and a neural network may be used as the same meaning. A deep neural network (DNN) may refer to a neural network that includes a plurality of hidden layers in addition to the input and output layers. When the deep neural network is used, the latent structures of data may be determined. That is, potential structures of photos, text, video, voice, and music (e.g., what objects are in the picture, what the content and feelings of the text are, what the content and feelings of the voice are) may be determined. The deep neural network may include a convolutional neural network (CNN), a recurrent neural network (RNN), a restricted Boltzmann machine (RBM), a deep belief network (DBN), a Q network, a U network, a Siam network, and the like.

The convolutional neural network as a kind of deep neural network includes a neural network including a convolutional layer. The convolutional neural network is a type of multilayer perceptrons designed to use minimal preprocessing. The CNN may be constituted by one or multiple convolutional layers and artificial neural network layers combined therewith. The CNN may additionally utilize a weight and pooling layers. Such a structure allows the CNN to fully utilize the input data of a two-dimensional structure. The convolutional neural network may be used for recognizing an object in an image. The convolutional neural network may represent and process image data as a matrix having the dimension. For example, in the case of image data encoded in red-green-blue (RGB), the image data may be represented as a two-dimensional (for example, a two-dimensional image) matrix for each of R, G, and B colors. That is, a color value of each pixel of the image data may become a component of the matrix and a size of the matrix may be equal to the size of the image. Accordingly, the image data may be represented by three two-dimensional matrix (three-dimensional data array).

In the convolutional neural network, matrix components at respective positions of a convolutional filter and the image are multiplied by each other while moving the convolutional filter to perform a convolutional process (input/output of a convolutional layer). The convolutional filter may be configured in a matrix in the form of n*n. The convolutional filter may be generally configured by a filter of a fixed form smaller than the total number of pixels of the image. In other words, when m*m images are input by the convolutional layer (e.g., a convolutional layer in which the size of the convolutional filter is n*n), a matrix representing n*n pixels including each pixel of the image may be a component multiplication with the convolutional filter (e.g., a multiplication of respective components of the matrix). Components matched with the convolutional filter may be extracted from the image by the multiplication with the convolutional filter. For example, a 3*3 convolutional filter for extracting a vertical straight-line component from the image may be constituted by [[0,1,0], [0,1,0], [0,1,0]]. When the 3*3 convolutional filter for extracting the vertical straight-line component from the image is applied to an input image, vertical straight-line components which match the convolutional filter may be extracted and outputted from the image. The convolutional layer may apply the convolutional filter to respective matrixes (e.g., R, G, and B colors in the case of R, G, and B coding images) for respective channels representing the image. The convolutional layer may extract features matched with the convolutional filter from the input image by applying the convolutional filter to the input image. A filter value (e.g., a value of each component of the matrix) of the convolutional filter may be updated by back propagation during a training process of the convolutional neural network.

A subsampling layer is connected to the output of the convolutional layer to simplify the output of the convolutional layer, thereby reducing a memory usage and a computational amount. For example, when the output of the convolutional layer is input to a pooling layer having a 2*2 max pooling filter, a maximum value included in each patch is output every 2*2 patches in each pixel of the image to compress the image. The aforementioned pooling may be a method that outputs a minimum value in the patch or outputs an average value of the patch and a predetermined (or selected) pooling method may be included in the present disclosure.

The convolutional neural network may include one or more convolutional layers and subsampling layers. The convolutional neural network repeatedly performs the convolutional process and a subsampling process (e.g., the aforementioned max pooling) to extract the features from the image. The neural network may extract global features of the image through the repeated convolutional process and subsampling process.

The output of the convolutional layer or the subsampling layer may be input to a fully connected layer. The fully connected layer is a layer in which all neurons in one layer and all neurons in an adjacent layer are connected. The fully connected layer may mean a structure in which all nodes of each layer are connected to all nodes of another layer in the neural network.

In an embodiment of the present disclosure, the neural network may include a deconvolutional neural network (DCNN) in order to perform segmentation of the medical data. The deconvolutional neural network performs a similar operation as calculating the convolutional neural network in a reverse direction. The deconvolutional neural network may output the features extracted from the convolutional neural network to a feature map related to original data. A description of a detailed configuration for the convolutional neural network is discussed in more detail in U.S. Pat. No. 9,870,768B2, the entire contents of which are incorporated herein by reference in this application.

The processor 120 may calculate at least some regions included in the medical data by using the diagnosis model. The processor 120 may output a score value for each of two or more classes with respect to at least some regions included in the medical data by using the diagnosis model. The score value for the class may be a probability of belonging to the corresponding class. The processor 120 may output the score value for each class with respect to each of the first region and the second region of the medical data by calculating the medical data. For example, by calculating one lung CT image, the score value for each class for the first region included in the corresponding image and the score value for each class for the second region may be separately outputted. The first region and the second region may each represent a different lesion. The first region and the second region may correspond to different lesions according to the score value output for each class, respectively. The detailed description of the calculation of the diagnosis model is just an example and the present disclosure is not limited thereto.

The processor 120 may determine the calculated region as one class by using the calculation result of the diagnosis model. The processor 120 may determine the calculated region as one class by using the score values for two or more classes, which are outputted from the diagnosis model. When the processor 120 determines the calculated region as one class, the processor 120 may classify the lesion as one finding corresponding to one class.

When the lesion is classified as one finding, the processor 120 may determine the corresponding lesion as the certain lesion.

The processor 120 may determine the finding of the corresponding region as a class having a largest score value among the score values for two or more classes. In addition, when the number of score [symbol] s having a first threshold or more is one, the processor 120 may determine the finding of the corresponding region as one. The certain lesion may be a case where the finding of the calculated region is determined as one.

The first threshold may be a threshold in which the calculated region may be classified as the corresponding class (e.g., finding). The processor 120 may determine that the calculated region belongs to a class corresponding to a score value of the first threshold or more. The processor 120 may determine that the calculated region does not belong to a class corresponding to a score value less than the first threshold. That is, only when the number of classes having the score value of the first threshold or more is one, the calculated region may be classified as one finding. When the number of classes having the score value of the first threshold or more is two or more, the calculated region may not be classified as one finding and may be classified as two or more findings.

For example, a result (class (score)) of calculating one region of the lung CT image by the diagnosis model may be Consolidation (0.9), Interstitial opacity (0.4), Nodule (0.1), and Atelectasis (0.2). Since the score value of consolidation is significantly larger than score values for other classes, the processor 120 may determine the finding of the corresponding region as consolidation. Specifically, since the score value for the consolidation class is largest among the score values, and the class having a score value of a first threshold (e.g., 0.7) or more is only one consolidation class, the processor 120 may determine the finding of the corresponding region as one consolidation class. Accordingly, the processor 120 may determine the finding of the corresponding lesion as consolidation. The processor 120 may determine the corresponding lesion as the certain lesion. The detailed description of the calculation of the medical data is just an example and the present disclosure is not limited thereto.

It may be difficult to determine the calculated region as one class through score values for two or more classes included in the calculation result of the diagnosis model. When it is difficult to determine the calculated region as one class, a detection result for the calculated region may be uncertain. The processor 120 may determine at least regions as the uncertain lesion when at least some regions are not determined as one class based on the score values for two or more classes included in the result of the calculation. Alternatively, the processor 120 may determine at least some regions as the uncertain lesion when at least some regions are determined as two or more classes based on the score values for two or more classes included in the result of the calculation.

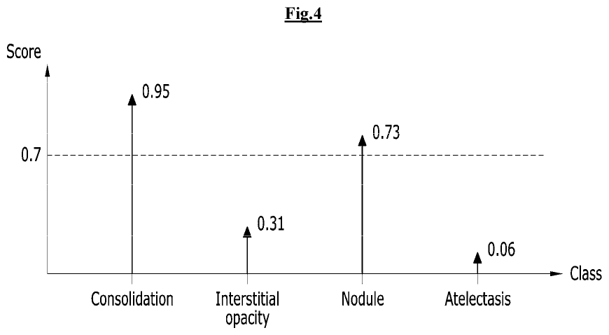

According to an embodiment of the present disclosure, the processor 120 may determine at least some regions as the uncertain lesion when the number of classes having a score value of a first threshold or more is 2 or more. Hereinafter, the uncertain lesion will be described with reference to FIG. 4. The processor 120 may check the score value of each of two or more classes for the calculated region. The processor 120 may check the number of score values which are the first threshold or more. For example, the first threshold may be 0.7 and an output for the calculated region may be Consolidation (0.95), Interstitial opacity (0.31), Nodule (0.73), and Atelectasis (0.06). The processor 120 may identify that two classes having the score value which is the first threshold or more are Consolidation and Nodule. The processor 120 may determine the finding of the corresponding lesion based on two classes having the score value which is the first threshold or more. The processor 120 may determine the finding of the corresponding lesion as Consolidation and Nodule. The processor 120 may determine that one region corresponds to a finding corresponding to two or more classes. The processor 120 may determine a lesion corresponding to two or more findings as the uncertain lesion. The detailed description of the uncertain lesion is just an example and the present disclosure is not limited thereto.

According to an embodiment of the present disclosure, the processor 120 may determine at least some regions as the uncertain lesion when there is no class having the score value of the first threshold or more. Only when the score value included in the calculation result is the first threshold or more, the processor 120 may determine that the corresponding region belongs to the class corresponding to the score. That is, when a score value less than the first threshold is derived, there is a low probability that the corresponding region will belong to the corresponding class, so the finding may not be determined as the class corresponding to the score value. When there is no class having the score value which is the first threshold or more, the processor 120 may determine that the finding corresponding to the corresponding lesion may not be determined. The processor 120 may determine the corresponding lesion as the uncertain lesion. For example, the first threshold may be 0.7 and an output for the calculated region may be Consolidation (0.65), Interstitial opacity (0.31), Nodule (0.6), and Atelectasis (0.06). The processor 120 may determine that there is no class having a value larger than 0.7. The processor 120 may determine the calculated region as the uncertain lesion. The detailed description of the uncertain lesion is just an example and the present disclosure is not limited thereto.

According to an embodiment of the present disclosure, when a difference between a largest score value and other score values is less than a threshold ratio or a threshold different value, the processor 120 may determine at least some regions as the uncertain lesion. When there is one overwhelmingly large score value, the processor 120 may classify the lesion into one class corresponding to the corresponding score value. However, when one class does not have the overwhelmingly large value, but has a similar value to another class, the processor 120 may determine that the corresponding lesion may not be classified into one finding. Even when there is only one class having the score value which is the first threshold or more, if there is no difference from score values of other classes by a threshold ratio or threshold difference value or more, the processor 120 may determine that the corresponding lesion corresponds to the uncertain lesion. When described as an example by referring to FIG. 4, the first threshold may be 0.7 and an output for the calculated region may be Consolidation (0.65), Interstitial opacity (0.31), Nodule (0.73), and Atelectasis (0.06). In the above case, Nodule has a score value of the first threshold or more as 0.73, but it may be determined that there is no significant degree of score value difference from Consolidation. For example, the threshold ratio may be 20%, and when comparing 0.73 which is the score value for the Nodule class with 0.65 which is the score value for the Consolidation class, the processor 120 may determine that there is no difference value of 20% or more between both score values. Alternatively, for example, the threshold difference value may be 0.1, and when comparing 0.73 which is the score value for the Nodule class with 0.65 which is the score value for the Consolidation class, the processor 120 may determine that there is no score value difference of 0.1 or more between both score values. Accordingly, when there is no significant degree of difference from score values of other classes, the processor 120 may determine the corresponding lesion as the uncertain lesion. The detailed description of the uncertain lesion is just an example and the present disclosure is not limited thereto.

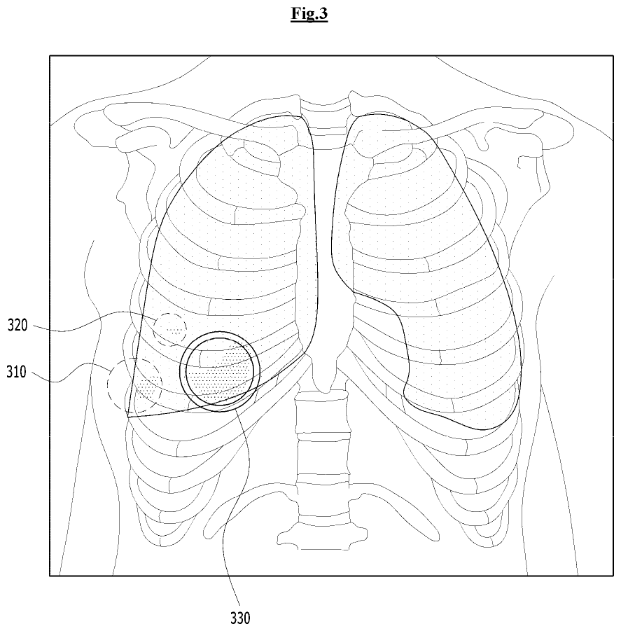

According to an embodiment of the present disclosure, the processor 120 may determine at least some regions as the uncertain lesion when a dispersion of the score values is less than a threshold dispersion value. The processor 120 may calculate the dispersion of the score values. When the dispersion of the score values is large, the large dispersion may indicate that score values for each class are unevenly distributed. When the score value of one class is overwhelmingly large, the processor 120 may class the corresponding lesion into one finding. When the processor 120 may determine the lesion as one class, the processor 120 may determine the corresponding lesion as the certain lesion. When the dispersion of the score values is small, the small dispersion may indicate that the score values for each class are evenly distributed. When the score value of one class is not overwhelmingly large, but score values of several classes are similar to each other, the processor 120 may class the corresponding lesion into a plurality of findings. When determining the lesion as a plurality of findings, the processor 120 may determine the corresponding lesion as the uncertain lesion. For example, in the case of FIGS. 3 and 4, it can be seen that among four classes, two classes have a similar value. That is, since one class does not have an overwhelmingly large value, the dispersion of the score values for the corresponding classes may be less than a threshold dispersion value. When the processor 120 may not classify the corresponding lesion into one finding, the processor 120 may determine the corresponding lesion as the uncertain lesion. The detailed description of the uncertain lesion is just an example and the present disclosure is not limited thereto.

Hereinafter, a method for determining a finding for the uncertain lesion will be described.

According to an embodiment of the present disclosure, when at least some regions are determined as two or more classes based on the score values for two or more classes included in the result of the calculation, the processor 120 may determine the findings corresponding to two or more classes as two or more findings of the uncertain lesion. When the number of score values which are a first threshold or more for the lesion is 2 or more, the processor 120 may determine the number of findings of the lesion as 2 or more. For example, in the case of FIG. 3, two classes having a score value larger than the first threshold are Consolidation and Nodule. The processor 120 may determine the finding of the corresponding lesion as Consolidation and Nodule. That is, when there are two or more classes having the score value which is the first threshold or more, the processor 120 may not determine the finding to correspond to one class having a maximum score value. A lesion readings result displaying method according to an embodiment of the present disclosure may not only provide only one finding corresponding to the maximum score method to the user but also provide, where there are uncertain findings, the corresponding findings to the user together. The users may check even uncertain findings together in addition to just checking only a finding having a highest possibility. The lesion readings result displaying method according to an embodiment of the present disclosure provides even the uncertain findings to the user together to more efficiently and more accurately determine a condition of a patient. The detailed description of the finding determining method is just an example and the present disclosure is not limited thereto.

According to an embodiment of the present disclosure, when at least some regions are not determined as one class, the processor 120 may determine findings corresponding to classes having a score value which is a predetermined (or selected) second threshold or more as one or more findings of the uncertain lesion. When the processor 120 may not class the lesion into one class, the processor 120 may determine findings in which there is a classification possibility for the corresponding lesion and display the determined findings on a user interface. The second threshold may be a value smaller than the first threshold. The second threshold may be a threshold for a case where there is a possibility that the corresponding lesion can be classified into the finding corresponding to the corresponding class. For example, the first threshold may be 0.7 and an output for the calculated region may be Consolidation (0.65), Interstitial opacity (0.31), Nodule (0.68), and Atelectasis (0.06). In the above case, since there is no score value larger than 0.7, the corresponding lesion may not be classified into one class. In the above example, the second threshold may be 0.5. The processor 120 may determine, as the findings of the uncertain lesion, Consolidation and Nodule which are the classes having the score value larger than the second threshold. That is, the lesion may not clearly be classified into one class, but the processor 120 may display findings having a certain degree of classification possibility on the user interface. The detailed description of the uncertain lesion is just an example and the present disclosure is not limited thereto.

According to an embodiment of the present disclosure, when at least some regions are not determined as one class, the processor 120 may determine findings corresponding to at least one of classes having a predetermined (or selected) upper number of score values as one or more findings of the uncertain lesion. For example, the processor 120 may determine a class corresponding to upper three score values as the finding. For example, an output for the calculated region may be Consolidation (0.68), Interstitial opacity (0.54), Nodule (0.6), and Atelectasis (0.06). When there is no score value which is the first threshold (0.7) or more, the processor 120 may determine that the lesion may not be classified into one finding. The processor 120 may determine the classes having upper three score values as the finding for the lesion. The processor 120 may determine Consolidation, Interstitial opacity, and Nodule as the finding of the uncertain lesion. The detailed description of the uncertain lesion is just an example and the present disclosure is not limited thereto.

According to an embodiment of the present disclosure, the processor 120 may receive a user input for adjusting a determination criterion of the uncertain lesion. The processor 120 may display a user interface for changing the determination criterion of the uncertain lesion. For example, the processor 120 may receive a user input for adjusting the first threshold, the second threshold, a predetermined (or selected) upper number, etc., which become criteria for determining the uncertain lesion. That is, when the first threshold is set to be low, too many uncertain lesions may be displayed. Therefore, the user adjusts the first threshold to be higher to display less uncertain lesions. The user may adjust the determination criterion of the uncertain lesion according to a read difficulty of the medical data. For example, in the case of CT that is obtained by photographing a knee having a low read difficulty, the criterion of the uncertain lesion may be set to be high (e.g., adjust the first threshold to be high). Therefore, only lesions having very high uncertainty may be displayed as the uncertain lesion. Alternatively, for example, in the case of CT obtained by photographing a brain having a high read difficulty, the criterion of the uncertain lesion may be set to be lower. In the case of brain CT, learning data itself may be small and when even a small lesion is ignored, a fatal impact may be exerted. Accordingly, in the case of the brain CT, the threshold is adjusted to be low and if there is uncertainty at all, an evidence for the corresponding uncertainty may be displayed on the medical data. The detailed description of the determination of the uncertain lesion is just an example and the present disclosure is not limited thereto.

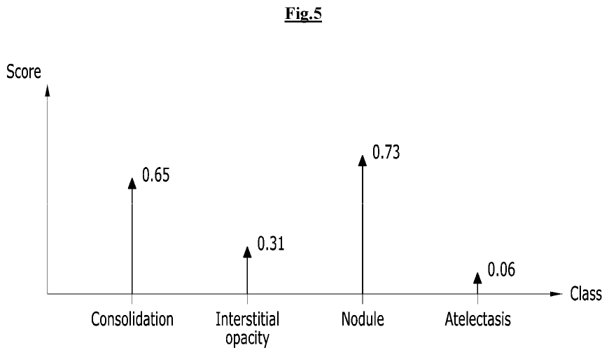

According to an embodiment of the present disclosure, the processor 120 may distinguish and display the certain lesion and the uncertain lesion. FIG. 5 illustrates an user interface that distinguishes and displays certain lesions 310 and 320, and an uncertain lesion 330. The processor 120 may display lesion information by various schemes as described above. The processor 120 may display the certain lesion and the uncertain lesion by different schemes. For example, the processor 120 may display the certain lesion with a dotted line, and the uncertain lesion with a double line. That is, the processor 120 displays the uncertain lesion by different schemes to allow the users to check the uncertain lesion once again. The detailed description of the lesion information displaying method is just an example and the present disclosure is not limited thereto.

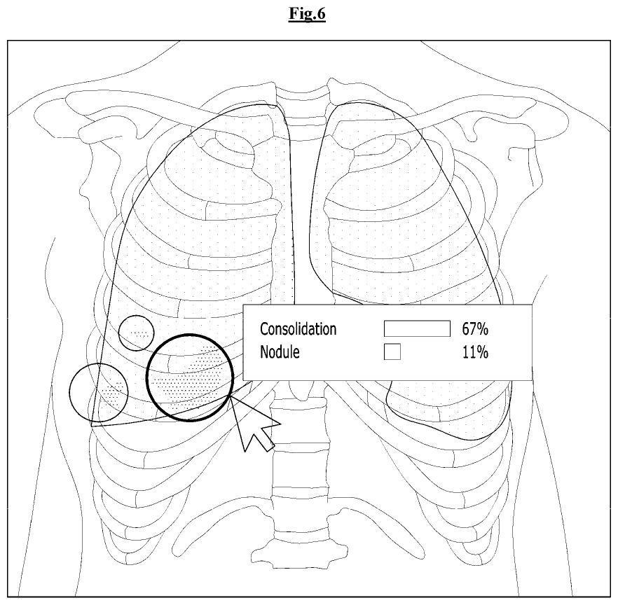

The processor 120 may display one or more findings related to the lesion information on the user interface in response to a user interaction with the lesion information. According to an embodiment of the present disclosure, the user interaction may include all types of user inputs inputted through the input unit 150. For example, the user interaction may include an operation of putting a mouse at a specific region displayed on the user interface or clicking the mouse. The detailed description of the user interaction is just an example and the present disclosure is not limited thereto. When described as an example by referring to FIG. 6, the processor 120 may identify that the mouse is positioned on the lesion information. When the mouse is positioned on the lesion information, the processor 120 may display the corresponding lesion by changing a display scheme of the corresponding lesion. For example, when the lesion information is displayed with the double line and the mouse is positioned on the corresponding lesion information, the lesion information may be displayed by changing the double line to a thick single line. When the mouse is positioned on the lesion information, the processor 120 may display one or more findings related to the lesion information. The processor 120 may display one or more findings related to the lesion information, for example, next to the lesion in a pop-up form. The processor 120 may display one finding for the certain lesion. The processor 120 may display one or more findings for the uncertain lesion. That is, the processor 120 may display one or more findings into which the corresponding lesion is likely to be classified on the user interface with respect to the uncertain lesion. The detailed description of the finding display is just an example and the present disclosure is not limited thereto.

The processor 120 may temporarily display the finding for the lesion according to a first user interaction. For example, when recognizing that the mouse is located on the lesion, the processor 120 may display the finding for the lesion. In addition, when the processor 120 recognizes that the mouse is not located on the lesion, the processor 120 may not display the finding for the lesion. That is, only while the mouse is located on the lesion, the processor 120 may temporarily display the finding. The processor 120 may fixedly display the finding for the lesion according to a second user interaction. For example, when the user clicks on the lesion, the processor 120 may display the finding for the lesion. In this case, after clicking on the lesion, even when the mouse is not located on the lesion, the finding may be displayed on the user interface. When the processor 120 receives the second user interaction with the lesion again, the processor 120 may prevent the finding from being displayed on the user interface. The detailed description of the finding display is just an example and the present disclosure is not limited thereto.

According to an embodiment of the present disclosure, the processor 120 may display an uncertainty degree for one or more findings related to the uncertain lesion. The processor 120 may display an uncertainty degree determined according to a score value of a class corresponding to one or more findings, for one or more findings related to the uncertain lesion. The uncertainty degree may mean a probability that the lesion can be classified into the corresponding finding. That is, as the uncertainty degree is larger, the probability that the lesion can be classified into the corresponding finding may be higher. As the score value of the class is larger, the processor 120 may display that the probability that the lesion can be classified into the finding corresponding to the corresponding class is high. When described for example by referring to FIG. 6, the lesion may not be classified into one finding, but may be classified into two findings, Consolidation and Nodule. The processor 120 may check score values corresponding to the classes Consolidation and Nodule, respectively. For example, when the score value for the Consolidation class is 0.68 and the score value for the Nodule class is 0.11, the processor 120 may display the uncertainty degree as Consolidation 67% and Nodule 11%. The uncertainty degree may be displayed by various schemes. For example, the uncertainty degree may be numerically displayed or schematized and displayed. For example, the uncertainty degree may be displayed as a percent or a value based on the score value. Alternatively, for example, the uncertainty degree may be displayed in a bar graph form based on the score value. For example, for a finding having a high uncertainty degree, a bar graph may be displayed to be longer and for a finding having a low uncertainty degree, the bar graph may be displayed to be shorter. The detailed description of the finding display is just an example and the present disclosure is not limited thereto.

When a score value for a predetermined (or selected) class, which is included in a result of calculating at least some regions included in the medical data by using the diagnosis model is equal to or greater than a third threshold, the processor 120 may display a finding corresponding to the predetermined (or selected) class. The predetermined (or selected) class may be determined according to user setting. For example, the predetermined (or selected) class may include a class having a large clinical meaning. The class having the large clinical meaning may include, for example, a case where is fatal for the patient, a case which is a rare case, but exerts a large influence on the patient, a class having a large clinical meaning when being combined with other findings, and the like. For example, the predetermined (or selected) class may be a class corresponding to a rare cancer. The third threshold may be a value smaller than the first threshold or the second threshold. In the case of the rare cancer, even though the score value itself is small, if the score value becomes the third threshold or more, the processor 120 may display a finding for the rare cancer on the user interface. That is, even when there is only a minor possibility that the rare cancer will occur, the possibility is separately notified to the user, thereby enhancing diagnosis accuracy for the patient. The detailed description of the finding display is just an example and the present disclosure is not limited thereto.

According to an embodiment of the present disclosure, the processor 120 may display the lesion information by different schemes according to the clinical meaning of the lesion. The clinical meaning may mean a meaningful result for patient diagnosis. For example, when finding A and finding B are detected in one lesion, it may be determined that the condition of the patient is very dangerous and has the clinical meaning. Alternatively, when it is not general that finding A and finding B are detected in one lesion, the processor 120 may determine that there is the clinical meaning. The processor 120 may display the lesion information by a different scheme from other lesion information. The detailed description of the display of the lesion information is just an example and the present disclosure is not limited thereto.

According to an embodiment of the present disclosure, the processor 120 may display the lesion information by different schemes according to the uncertainty degree of the lesion. The processor 120 may determine the uncertainty degree of the lesion according to the score value for each class for the lesion. For example, when two or more findings are detected for the lesion, and there is no significant difference between score values for classes of two or more findings, respectively, an uncertainty degree for which finding is correct may be larger. Therefore, when there are two or more classes in which there is no significant difference between the score values, the processor 120 may display the corresponding lesion information to be distinguished from other lesion information. The detailed description of the display of the lesion information is just an example and the present disclosure is not limited thereto.

According to an embodiment of the present disclosure, the processor 120 may post-process and display the lesion information. The post-process may be to correct the lesion of the medical data to be better shown by comparison with other regions. The post-process may include, for example, adjustment such as noise removal for the lesion, brightness, comparison, contrast, sharpening, windowing, etc. The concrete description of the post-process method is just an example and the present disclosure is not limited thereto.

The post-process method may be determined according to the user selection input. For example, the user may determine the post-process method by different schemes separately for a plurality of lesion regions. For example, according to the user selection input, sharpening processing may be further performed for first lesion information and second lesion information may be displayed by adjusting the brightness. That is, the users may manually adjust the corresponding lesion to be better shown while checking the lesion readings result. The concrete description of the post-process method is just an example and the present disclosure is not limited thereto.

The post-process method may be determined by at least one of a display of a lesion, a comparison between a lesion and a region around the lesion, or a type of findings corresponding to a lesion. The processor 120 may determine the post-process method according to a display degree of the lesion itself. For example, when the display degree of the lesion itself is less than a threshold brightness, comparison, and noise, the processor 120 may perform a post-process to correspond to the corresponding threshold. For example, when the brightness of the lesion is less than a threshold brightness, the processor 120 may adjust the lesion to a threshold brightness or more. The processor 120 compares the lesion and a lesion peripheral region and when the lesion has lower visibility than the lesion peripheral region, the processor 120 may perform the post-process so as to be shown better. For example, when the lesion has much lower brightness than the lesion peripheral region, the brightness may be adjusted and displayed to be higher. The processor 120 may perform the post-process by a prestored scheme according to the finding. For example, finding A may be stored in advance to be post-processed by scheme A and finding B may be stored in advance to be post-processed by scheme B. The processor 120 may determine the post-process method according to one or more findings determined for the lesion. The concrete description of the post-process method is just an example and the present disclosure is not limited thereto.

The processor 120 may post-process and display at least one lesion information displayed in the medical data in response to the user interaction. The processor 120 may identify that a user input such as the mouse is located in the lesion information. The processor 120 may post-process and display the lesion information while the user interaction is performed for the lesion information (for example, for a time interval in which the mouse is located on the lesion information). That is, the user may be configured to select and post-process some lesion information among a plurality of lesion information displayed on the medical data. The concrete description of the post-process method is just an example and the present disclosure is not limited thereto.

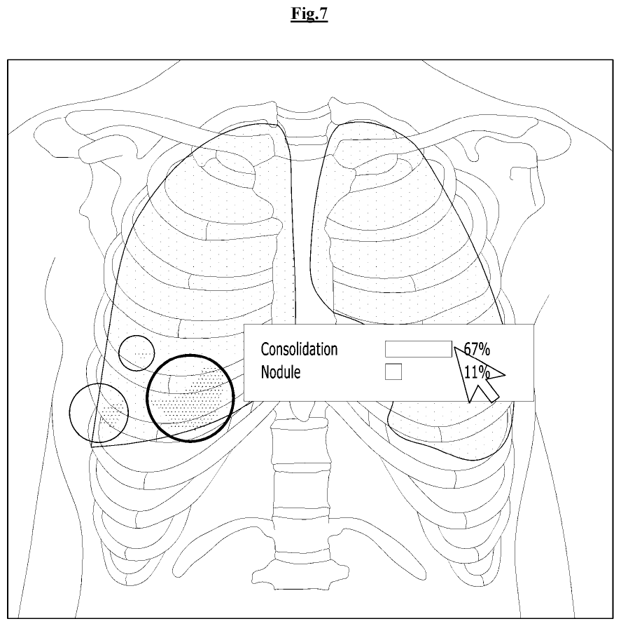

According to an embodiment of the present disclosure, the processor 120 may generate a reading for a finding subjected to a user selection input in response to a user selection input for at least one finding related to the lesion information. When described for example by referring to FIG. 7, when a user selection input for at least one finding, a reading text for the corresponding finding may be generated. For example, two findings Consolidation and Nodule may be displayed for one lesion. The processor 120 may receive a user selection input for Consolidation of two findings. The processor 120 may generate a read sentence based on finding Consolidation. That is, the processor 120 may generate the read sentence to include a diagnosis result based on Consolidation. The detailed description of the generation of the read sentence is just an example and the present disclosure is not limited thereto.

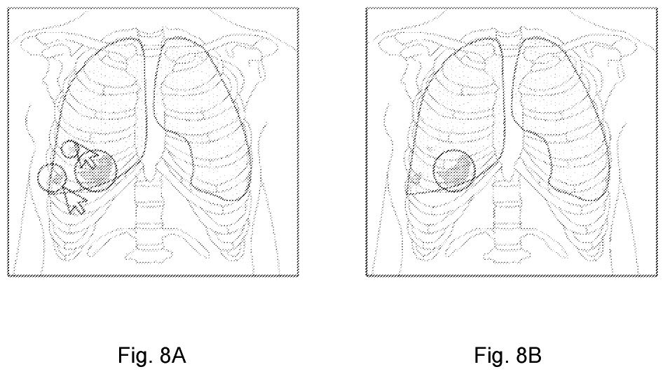

According to an embodiment of the present disclosure, the processor 120 may not display the lesion information in response to a user deletion input for the lesion information. The processor 120 may delete the lesion information on which the user clicks among two or more lesion information displayed in the medical data on the user interface. When described by referring to FIGS. 8A and 8B, the lesion information on which the user clicks among two or more lesion information displayed in FIG. 8A may be deleted from the user interface and displayed as illustrated in FIG. 8B. The users may delete lesion information which is wrongly displayed or is not significant among the lesion information displayed on the medical data. The processor 120 may a user input of deleting at least some lesion information when retraining the diagnosis model. For example, the lesion information deleted by the user is determined to have an error in an output of the diagnosis model to reflect such an error to the learning data and to be used for retraining the diagnosis model. The detailed description of the display of the lesion information is just an example and the present disclosure is not limited thereto.

According to an embodiment of the present disclosure, the processor 120 may display additional information for the lesion information. The additional information may include information for assisting determination of the user for the lesion. The processor 120 may display additional information for assisting determination of the user for medical data on the user interface while displaying a reading result for the medical data. For example, the additional information may include at least one of patient information, history information, other medical information, or reference case information.

The patient information may mean basic information of the patient corresponding to the medical data. For example, the patient information may include age, gender, and the like of the patient. The detailed description of the patient information is just an example and the present disclosure is not limited thereto.

The history information may be information on past medical data generated at a different time from the medical data. For example, when the medical data is an X-RAY image of patient A in August, the past medical data may be X-RAY for patient A in January. That is, the processor 120 provides history information for comparing a past examination image and a current examination image of the patient to assist medical data reading of a medical team. For example, in the case of malignant tumors, a case where the malignant tumor decreases in size and a case where the malignant tumor increases in size as compared with the past may be different in surgery or therapy. The processor 120 may compare the lesion included in the medical data and the lesion included in the past medical data. The processor 120 may display additional information including the comparison result on the user interface. The processor 120 may provide, to the user interface, a quantitative or qualitative comparison result between both medical data, for example. For example, the processor 120 may just display the past medical data on the user interface together. Alternatively, when at least some lesions are changed, the processor 120 may display a changed degree on the user interface. When comparing with the past medical data, if the lesion is significantly changed, the processor 120 may generate a separate notification. The detailed description of the history information is just an example and the present disclosure is not limited thereto.

Other medical information may mean various other information related to the medical data. Other medical information may be at least one diagnosis or examination result stored for the patient. For example, when the medical data is a CT image of the patient, other medical information may include a blood examination result, an ultrasonic examination result, and the like of the user. The detailed description of the other medical information is just an example and the present disclosure is not limited thereto.

The reference case information may be related to a lesion similar to a lesion corresponding to the lesion information. The processor 120 may compare the lesion information included in the medical data and lesion information for plurality of medical data stored in a database. The processor 120 may identify another medical data having a similar lesion. The processor 120 may identify another medical data having a feature similar to a feature of the lesion included in the medical data, for example. The processor 120 may extract case information of the patient corresponding to another medical data. The case information may include the patient information, a diagnosis of the patient or examination information, prognosis information, and the like. That is, a case of another patient having a similar lesion is provided to the user at the time of reading the medical data to assist reading of the corresponding medical data. The detailed description of the reference case information is just an example and the present disclosure is not limited thereto.

A computing device 100 for displaying a lesion readings result according to an embodiment of the present disclosure may include a network unit 110, a processor 120, a memory 130, an output unit 140, and an input unit 150.

The network unit 110 may transmit and receive medical data according to an embodiment of the present disclosure to and from other computing devices, servers, and the like. In addition, the network unit 110 may enable communication among a plurality of computing devices so that operations for lesion reading or model learning are distributively performed in each of the plurality of computing devices. The network unit 110 enables communication among a plurality of computing devices so that a calculation for the lesion reading or model learning using the network function is distributively performed.

The network unit 110 according to an embodiment of the present disclosure may operate based on arbitrary type wired/wireless communication technology which is currently used and implemented, such as local area (short range), long range, wired, and wireless, and may be used even in other networks.

The processor 120 may be constituted by one or more cores and may include processors for learning a model, which include a central processing unit (CPU), a general purpose graphics processing unit (GPGPU), a tensor processing unit (TPU), and the like of the computing device. The processor 120 may read a computer program stored in the memory 130 to provide a lesion readings result according to an embodiment of the present disclosure. According to the embodiment of the present disclosure, the processor 120 may perform a calculation for providing the lesion readings result.

The memory 130 may store a computer program for providing the lesion readings result according to an embodiment of the present disclosure and the stored computer program may be read and driven by the processor 120.

The memory 130 according to the embodiments of the present disclosure may store a program for a motion of the processor 120 and temporarily or permanently store input/output data or events. The memory 130 may store data regarding the display and the sound. The memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk.

The output unit 140 according to an embodiment of the present disclosure may display a user interface (UI) for providing a lesion readings result. The output unit 140 may display the user interface illustrated in FIGS. 2, and 5 to 8. The user interfaces illustrated in the figures and described above are just examples and the present disclosure is not limited thereto.

The output unit 140 according to an embodiment of the present disclosure may output any type of information created or determined by the processor 120 or any type of information received by the network unit 110.

The output unit 140 according to an embodiment of the present disclosure may include at least one of a liquid crystal display (LCD), a thin film transistor-liquid crystal display (TFT LCD), an organic light-emitting diode (OLED), a flexible display, and a 3D display. Some display modules among them may be configured as a transparent or light transmissive type to view the outside through the displays. This may be called a transparent display module and a representative example of the transparent display module includes a transparent OLED (TOLED), and the like.

User input may be received through the input unit 150 according to an embodiment of the present disclosure. The input unit 150 according to an embodiment of the present disclosure may include keys and/or buttons on the user interface or physical keys and/or buttons for receiving the user input. A computer program for controlling a display according to embodiments of the present disclosure may be executed according to the user input through the input unit 150.

The input unit 150 according to embodiments of the present disclosure receives a signal by sensing a button operation or a touch input of the user or receives speech or a motion of the user through a camera or a microphone to convert the received signal, speech, or motion into an input signal. To this end, speech recognition technologies or motion recognition technologies may be used.

The input unit 150 according to embodiments of the present disclosure may be implemented as external input equipment connected to the computing device 100. For example, the input equipment may be at least one of a touch pad, a touch pen, a keyboard, or a mouse for receiving the user input, but this is just an example and the present disclosure is not limited thereto.

The input unit 150 according to an embodiment of the present disclosure may recognize user touch input. The input unit 150 according to an embodiment of the present disclosure may be the same component as the output unit 140. The input unit 150 may be configured as a touch screen implemented to receive selection input of the user. The touch screen may adopt any one scheme of a contact type capacitive scheme, an infrared light detection scheme, a surface acoustic wave (SAW) scheme, a piezoelectric scheme, and a resistance film scheme. A detailed description of the touch screen is just an example according to an embodiment of the present disclosure and various touch screen panels may be adopted in the computing device 100. The input unit 150 configured as the touch screen may include a touch sensor. The touch sensor may be configured to convert a change in pressure applied to a specific portion of the input unit 150 or capacitance generated at the specific portion of the input unit 150 into an electrical input signal. The touch sensor may be configured to detect touch pressure as well as a touched position and area. When there is a touch input for the touch sensor, a signal(s) corresponding to the touch input is(are) sent to a touch controller. The touch controller processes the signal(s) and thereafter, transmits data corresponding thereto to the processor 120. As a result, the processor 120 may recognize which area of the input unit 150 is touched, and the like.

In an embodiment of the present disclosure, a server may include other components for performing a server environment of the server. The server may include all arbitrary types of devices. The server as a digital device may be a digital device with a calculation capability, which has a processor installed therein and a memory, such as a laptop computer, a notebook computer, a desktop computer, a web pad, or a mobile phone.

A server (not illustrated) performing an operation for providing the user terminal with the user interface for displaying the lesion readings result according to an embodiment of the present disclosure may include a network unit, a processor, and a memory.

The server may generate the user interface according to embodiments of the present disclosure. The server may be a computing system providing information to a client (e.g., user terminal) through a network. The server may transmit the generated user interface to the user terminal. In this case, the user terminal may be an arbitrary type of computing device 100 which may access the server. The processor of the server may transmit the user interface to the user terminal through the network unit. The server according to embodiments of the present disclosure may be, for example, a cloud server. The server may be a web server that processes a service. A type of server described above is just an example and the present disclosure is not limited thereto.

The network unit, the processor, and the memory included in the server according to embodiments of the present disclosure may play the same roles or be configured similarly as the network unit 110, the processor 120, and the memory 130 included in the computing device 100.

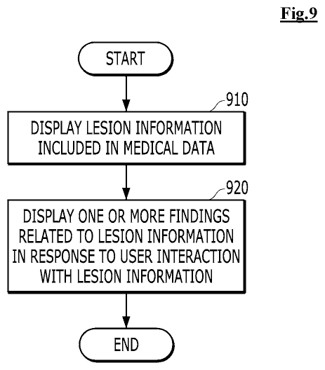

FIG. 9 is a flowchart for showing a lesion readings result according to an embodiment of the present disclosure.

The computing device 100 may display lesion information included in medical data (910).

The computing device 100 may display lesion information for at least one of a certain lesion or an uncertain lesion included in the medical data. The certain lesion may be a lesion in which at least some regions included in the medical data are classified as one finding. The uncertain lesion may be a lesion in which at least some regions included in the medical data are not classified as one finding.

The computing device 100 may calculate at least some regions included in the medical data by using a diagnosis model including one or more network functions. The computing device 100 may determine at least regions as the uncertain lesion when at least some regions are not determined as one class based on the score values for two or more classes included in the result of the calculation.

The computing device 100 may determine at least some regions as the uncertain lesion when the number of classes having a score value of a first threshold or more is 2 or more. The computing device 100 may determine at least some regions as the uncertain lesion when there is no class having the score value of the first threshold or more. When a difference between a largest score value and other score values is less than a threshold ratio or a threshold different value, the computing device 100 may determine at least some regions as the uncertain lesion. The computing device 100 may determine at least some regions as the uncertain lesion when a dispersion of the score values is less than a threshold dispersion value.

When at least some regions are determined as two or more classes based on the score values for two or more classes included in the result of the calculation, the computing device 100 may determine two or more findings of the uncertain lesion as the findings corresponding to two or more classes.

When at least some regions are not determined as one class based on score values for two or more classes included in a result of a calculation, the computing device 100 may determine one or more findings of the uncertain lesion as findings corresponding to at least one of a class having a score value which is a predetermined (or selected) second threshold or more or a class having a predetermined (or selected) upper number of score values.

The computing device 100 may distinguish and display the certain lesion and the uncertain lesion.

The computing device 100 may display one or more findings related to the lesion information in response to the user interaction with the lesion information (920).

The computing device 100 may display an uncertainty degree for one or more findings related to the uncertain lesion. The computing device 100 may display an uncertainty degree determined according to a score value of a class corresponding to one or more findings, for one or more findings related to the uncertain lesion.

When a score value for a predetermined (or selected) class, which is included in a result of calculating at least some regions included in the medical data by using the diagnosis model is equal to or greater than a third threshold, the computing device 100 may display a finding corresponding to the predetermined (or selected) class.

The computing device 100 may display the lesion information by different schemes according to at least one of a clinical meaning of the lesion information or an uncertainty degree of the lesion information.

The computing device 100 may post-process and display the lesion information. The post-process may be determined according to a user selection input or by at least one of a display of a lesion, a comparison between a lesion and a region around the lesion, or a type of findings corresponding to a lesion.

The computing device 100 may generate a reading text for a finding subjected to a user selection input in response to a user selection input for at least one finding related to the lesion information.

The computing device 100 may not display the lesion information in response to a user deletion input for the lesion information.

The computing device 100 may display additional information for the lesion information. The additional information may include information to aid in a judgment of a user about a lesion and at least one of patient information, history information, other medical information, or reference case information.

The lesion readings result according to an embodiment of the present disclosure may be implemented by a module, a circuit, a means, and a logic that perform the operation.

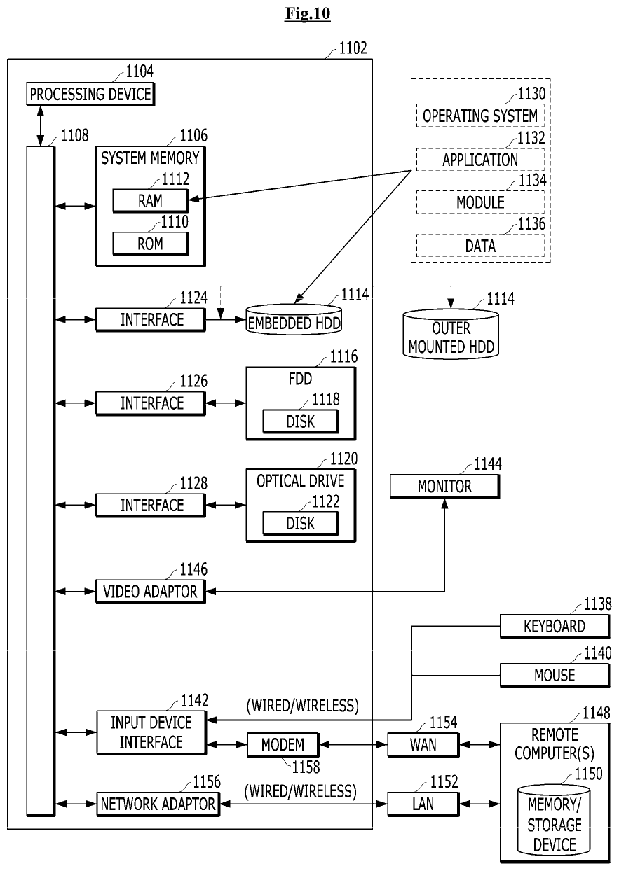

FIG. 10 is a simple and normal schematic view of an computing environment in which the embodiments of the present disclosure may be implemented.

It is described above that the present disclosure may be generally implemented by the computing device, but those skilled in the art will well know that the present disclosure may be implemented in association with a computer executable command which may be executed on one or more computers and/or in combination with other program modules and/or as a combination of hardware and software.

In general, the program module includes a routine, a program, a component, a data structure, and the like that execute a specific task or implement a specific abstract data type. Further, it will be well appreciated by those skilled in the art that the method of the present disclosure can be implemented by other computer system configurations including a personal computer, a handheld computing device, microprocessor-based or programmable home appliances, and others (the respective devices may operate in connection with one or more associated devices as well as a single-processor or multi-processor computer system, a mini computer, and a main frame computer.

The embodiments described in the present disclosure may also be implemented in a distributed computing environment in which predetermined (or selected) tasks are performed by remote processing devices connected through a communication network. In the distributed computing environment, the program module may be positioned in both local and remote memory storage devices.

The computer generally includes various computer readable media. Media accessible by the computer may be computer readable media regardless of types thereof and the computer readable media include volatile and non-volatile media, transitory and non-transitory media, and mobile and non-mobile media. As a non-limiting example, the computer readable media may include both computer readable storage media and computer readable transmission media. The computer readable storage media include volatile and non-volatile media, temporary and non-temporary media, and movable and non-movable media implemented by a predetermined (or selected) method or technology for storing information such as a computer readable instruction, a data structure, a program module, or other data. The computer readable storage media include a RAM, a ROM, an EEPROM, a flash memory or other memory technologies, a CD-ROM, a digital video disk (DVD) or other optical disk storage devices, a magnetic cassette, a magnetic tape, a magnetic disk storage device or other magnetic storage devices or predetermined (or selected) other media which may be accessed by the computer or may be used to store desired (or selected) information, but are not limited thereto.

The computer readable transmission media generally implement the computer readable command, the data structure, the program module, or other data in a carrier wave or a modulated data signal such as other transport mechanism and include all information transfer media. The term “modulated data signal” means a signal acquired by configuring or changing at least one of characteristics of the signal so as to encode information in the signal. As a non-limiting example, the computer readable transmission media include wired media such as a wired network or a direct-wired connection and wireless media such as acoustic, RF, infrared and other wireless media. A combination of any media among the aforementioned media is also included in a range of the computer readable transmission media.

An environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined (or selected) processor among various commercial processors. A dual processor and other multi-processor architectures may also be used as the processing device 1104.

The system bus 1108 may be any one of several types of bus structures which may be additionally interconnected to a local bus using any one of a memory bus, a peripheral device bus, and various commercial bus architectures. The system memory 1106 includes a read only memory (ROM) 1110 and a random access memory (RAM) 1112. A basic input/output system (BIOS) is stored in the non-volatile memories 1110 including the ROM, the EPROM, the EEPROM, and the like and the BIOS includes a basic routine that assists in transmitting information among components in the computer 1102 at a time such as in-starting. The RAM 1112 may also include a high-speed RAM including a static RAM for caching data, and the like.

The computer 1102 also includes an interior hard disk drive (HDD) 1114 (for example, EIDE and SATA), in which the interior hard disk drive 1114 may also be configured for an exterior purpose in an appropriate chassis (not illustrated), a magnetic floppy disk drive (FDD) 1116 (for example, for reading from or writing in a mobile diskette 1118), and an optical disk drive 1120 (for example, for reading a CD-ROM disk 1122 or reading from or writing in other high-capacity optical media such as the DVD, and the like). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. An interface 1124 for implementing an exterior drive includes at least one of a universal serial bus (USB) and an IEEE 1394 interface technology or both of them.

The drives and the computer readable media associated therewith provide non-volatile storage of the data, the data structure, the computer executable instruction, and others. In the case of the computer 1102, the drives and the media correspond to storing of predetermined (or selected) data in an appropriate digital format. In the description of the computer readable media, the mobile optical media such as the HDD, the mobile magnetic disk, and the CD or the DVD are mentioned, but it will be well appreciated by those skilled in the art that other types of media readable by the computer such as a zip drive, a magnetic cassette, a flash memory card, a cartridge, and others may also be used in an operating environment and further, the predetermined (or selected) media may include computer executable commands for executing the methods of the present disclosure.

Multiple program modules including an operating system 1130, one or more application programs 1132, other program module 1134, and program data 1136 may be stored in the drive and the RAM 1112. All or some of the operating system, the application, the module, and/or the data may also be cached in the RAM 1112. It will be well appreciated that the present disclosure may be implemented in operating systems which are commercially usable or a combination of the operating systems.

A user may input instructions and information in the computer 1102 through one or more wired/wireless input devices, for example, pointing devices such as a keyboard 1138 and a mouse 1140. Other input devices (not illustrated) may include a microphone, an IR remote controller, a joystick, a game pad, a stylus pen, a touch screen, and others. These and other input devices are often connected to the processing device 1104 through an input device interface 1142 connected to the system bus 1108, but may be connected by other interfaces including a parallel port, an IEEE 1394 serial port, a game port, a USB port, an IR interface, and others.

A monitor 1144 or other types of display devices are also connected to the system bus 1108 through interfaces such as a video adapter 1146, and the like. In addition to the monitor 1144, the computer generally includes other peripheral output devices (not illustrated) such as a speaker, a printer, others.

The computer 1102 may operate in a networked environment by using a logical connection to one or more remote computers including remote computer(s) 1148 through wired and/or wireless communication. The remote computer(s) 1148 may be a workstation, a computing device computer, a router, a personal computer, a portable computer, a micro-processor based entertainment apparatus, a peer device, or other general network nodes and generally includes multiple components or all of the components described with respect to the computer 1102, but only a memory storage device 1150 is illustrated for brief description. The illustrated logical connection includes a wired/wireless connection to a local area network (LAN) 1152 and/or a larger network, for example, a wide area network (WAN) 1154. The LAN and WAN networking environments are general environments in offices and companies and facilitate an enterprise-wide computer network such as Intranet, and all of them may be connected to a worldwide computer network, for example, the Internet.

When the computer 1102 is used in the LAN networking environment, the computer 1102 is connected to a local network 1152 through a wired and/or wireless communication network interface or an adapter 1156. The adapter 1156 may facilitate the wired or wireless communication to the LAN 1152 and the LAN 1152 also includes a wireless access point installed therein in order to communicate with the wireless adapter 1156. When the computer 1102 is used in the WAN networking environment, the computer 1102 may include a modem 1158 or has other means that configure communication through the WAN 1154 such as connection to a communication computing device on the WAN 1154 or connection through the Internet. The modem 1158 which may be an internal or external and wired or wireless device is connected to the system bus 1108 through the serial port interface 1142. In the networked environment, the program modules described with respect to the computer 1102 or some thereof may be stored in the remote memory/storage device 1150. It will be well known that an illustrated network connection is and other means configuring a communication link among computers may be used.

The computer 1102 performs an operation of communicating with predetermined (or selected) wireless devices or entities which are disposed and operated by the wireless communication, for example, the printer, a scanner, a desktop and/or a portable computer, a portable data assistant (PDA), a communication satellite, predetermined (or selected) equipment or place associated with a wireless detectable tag, and a telephone. This at least includes wireless fidelity (Wi-Fi) and Bluetooth wireless technology. Accordingly, communication may be a predefined structure like the network in the related art or just ad hoc communication between at least two devices.

The wireless fidelity (Wi-Fi) enables connection to the Internet, and the like without a wired cable. The Wi-Fi is a wireless technology such as the device, for example, a cellular phone which enables the computer to transmit and receive data indoors or outdoors, that is, anywhere in a communication range of a base station. The Wi-Fi network uses a wireless technology called IEEE 802.11(a, b, g, and others) in order to provide safe, reliable, and high-speed wireless connection. The Wi-Fi may be used to connect the computers to each other or the Internet and the wired network (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or operate in a product including both bands (dual bands).

It will be appreciated by those skilled in the art that information and signals may be expressed by using various different predetermined (or selected) technologies and techniques. For example, data, instructions, commands, information, signals, bits, symbols, and chips which may be referred in the above description may be expressed by voltages, currents, electromagnetic waves, magnetic fields or particles, optical fields or particles, or predetermined (or selected) combinations thereof.

It may be appreciated by those skilled in the art that various logical blocks, modules, processors, means, circuits, and algorithm steps described in association with the embodiments disclosed herein may be implemented by electronic hardware, various types of programs or design codes (for easy description, herein, designated as software), or a combination of all of them. In order to clearly describe the intercompatibility of the hardware and the software, various components, blocks, modules, circuits, and steps have been generally described above in association with functions thereof. Whether the functions are implemented as the hardware or software depends on design restrictions given to a specific application and an entire system. Those skilled in the art of the present disclosure may implement functions described by various methods with respect to each specific application, but it should not be interpreted that the implementation determination departs from the scope of the present disclosure.

Various embodiments presented herein may be implemented as manufactured articles using a method, an apparatus, or a standard programming and/or engineering technique. The term manufactured article includes a computer program, a carrier, or a medium which is accessible by a predetermined (or selected) computer-readable storage device. For example, a computer-readable storage medium includes a magnetic storage device (for example, a hard disk, a floppy disk, a magnetic strip, or the like), an optical disk (for example, a CD, a DVD, or the like), a smart card, and a flash memory device (for example, an EEPROM, a card, a stick, a key drive, or the like), but is not limited thereto. Further, various storage media presented herein include one or more devices and/or other machine-readable media for storing information.

It will be appreciated that a specific order or a hierarchical structure of steps in the presented processes is one example of accesses. It will be appreciated that the specific order or the hierarchical structure of the steps in the processes within the scope of the present disclosure may be rearranged based on design priorities. Appended method claims provide elements of various steps in a sample order, but the method claims are not limited to the presented specific order or hierarchical structure.

The description of the presented embodiments is provided so that those skilled in the art of the present disclosure use or implement the present disclosure. Various modifications of the embodiments will be apparent to those skilled in the art and general principles defined herein can be applied to other embodiments without departing from the scope of the present disclosure. Therefore, the present disclosure is not limited to the embodiments presented herein, but should be interpreted within the widest range which is coherent with the principles and new features presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A computer program stored in a computer readable storage medium wherein when the computer program is executed in one or more processors, the computer program provides a user interface for displaying a lesion readings result, the user interface comprising:
lesion information comprised in medical data; and
one or more findings related to the lesion information and displayed in response to a user interaction for the lesion information.

2. The computer program stored in a computer readable storage medium according to claim 1, wherein the lesion information comprised in medical data comprising:
lesion information for at least one of a certain lesion in which at least some regions included in the medical data are classified as one finding, or an uncertain lesion in which at least some regions included in the medical data are not classified as one finding.

3. The computer program stored in a computer readable storage medium according to claim 2, wherein the certain lesion and the uncertain lesion are distinguished and displayed.

4. The computer program stored in a computer readable storage medium according to claim 2, wherein the user interface further comprising:
a degree of uncertainty for each of one or more findings associated with the uncertain lesion, which is a probability that the uncertain lesion can be classified as a finding associated with the uncertain lesion.

5. The computer program stored in a computer readable storage medium according to claim 2, wherein when the at least some regions are calculated using a diagnostic model comprising one or more network functions, the uncertain lesion comprises the at least some regions, which are not determined as one class or are determined as two or more classes, based on score values for two or more classes included in a result of the calculation.

6. The computer program stored in a computer readable storage medium according to claim 5, wherein two or more findings of the uncertain lesion correspond to each of the two or more classes.

7. The computer program stored in a computer readable storage medium according to claim 5, wherein the one or more findings of the uncertain lesion correspond to at least one of a class having a score value equal to or greater than a selected second threshold value, or a class having a selected higher number of score values.

8. The computer program stored in a computer readable storage medium according to claim 2, wherein when the at least some regions are calculated using a diagnostic model comprising one or more network functions, and based on the score values for two or more classes included in the result of the calculation, the uncertain lesion is at least one of the following cases where there are two or more classes having a score value equal to or greater than a first threshold value, where there is no class having a score value equal to or greater than the first threshold value, where a difference between the largest score value and the other score values is less than a threshold ratio or a threshold difference value, or where a variance of score values is less than a threshold variance value.

9. The computer program stored in a computer readable storage medium according to claim 2, wherein when the at least some regions are calculated using a diagnostic model comprising one or more network functions, the uncertain lesion comprises the at least some regions, which are determined as one class, based on the score values for two or more classes included in a result of the calculation.

10. The computer program stored in a computer readable storage medium according to claim 2, wherein the user interface further comprising:
a degree of uncertainty for each of one or more findings associated with the uncertain lesion, which is determined according to a score value of a class corresponding to each of the one or more findings associated with the uncertain lesion.

11. The computer program stored in a computer readable storage medium according to claim 1, wherein the one or more findings related to the lesion information comprising:
a finding corresponding to a selected class when a calculating result of a diagnostic model for at least some regions included in the medical data has a score equal to or greater than a third threshold value for the selected class.

12. The computer program stored in a computer readable storage medium according to claim 1, wherein the lesion information is displayed in different ways depending on at least one of a clinical meaning of the lesion information or a degree of uncertainty of the lesion information.

13. The computer program stored in a computer readable storage medium according to claim 1, wherein the lesion information is post-processed lesion information, and wherein the post-process is determined according to a user selection input or by at least one of a display of a lesion, a comparison between a lesion and a region around a lesion, or a type of findings corresponding to a lesion.

14. The computer program stored in a computer readable storage medium according to claim 1, wherein the one or more processors perform an operation of generating a reading for a finding inputted by a user selection input in response to the user selection input for at least one finding related to the lesion information.

15. The computer program stored in a computer readable storage medium according to claim 1, wherein the user interface does not display the lesion information in response to a user deletion input for the lesion information.

16. The computer program stored in a computer readable storage medium according to claim 1, wherein the user interface further comprises additional information about the lesion information, and wherein the additional information comprises information to aid in a judgment of a user about a lesion and at least one of patient information, history information, other medical information, or reference case information.

17. The computer program stored in a computer readable storage medium according to claim 16, wherein the history information comprises a comparison result of one or more lesions included in past medical data generated at a time different from the medical data and one or more lesions included in the medical data.

18. A server, comprising:
a processor comprising one or more cores;
a network circuit; and
a memory,
wherein the processor is configured to:
determine to transmit a user interface to a user terminal through the network circuit,
wherein the user interface comprising:
lesion information comprised in medical data; and
one or more findings related to the lesion information which are displayed in response to a user interaction with the lesion information.

19. A user terminal, comprising:
a processor comprising one or more cores;
a memory, and
an output circuit providing a user interface,
wherein the user interface comprising:
lesion information comprised in medical data; and
one or more findings related to the lesion information which are displayed in response to a user interaction with the lesion information.
